(19) United States
(12) Patent Application Publication
(10) Pub. No.: US 2015/0110655 A1
(43) Pub. Date: Apr. 23, 2015

(54) LINEAR PERISTALTIC PUMP

(71) Applicant: PHYSIDIA, saint-Barthelemy-d'Anjou (FR)

(72) Inventors: Philippe Ferme, Le Plessis Grammoire (FR); Julien Marine, Angers (FR); Eric Vincent, Angers (FR)

(73) Assignee: PHYSIDIA, Saint-Barthelemy-d'Anjou (FR)

(21) Appl. No.: 14/402,754

(22) PCT Filed: May 21, 2013
(86) PCT No.: PCT/FR2013/051100

§ 371 Date: Nov. 21, 2014

(30) Foreign Application Priority Data
May 23, 2012 (FR) 1254691

(51) Int. Cl. F04B 43/12; F04B 49/06; A61M 1/36; F04B 43/08

(52) U.S. Cl. CPC F04B 43/1276; F04B 43/08; F04B 49/065; A61M 1/3663; A61M 2202/0021
USPC 417/477.6

(57) ABSTRACT

A peristaltic pump (1) including a tray, referred to as a pump body (3), which includes a substantially planar surface (32) against which a flexible tube (5) for the passage of fluid is intended to be positioned, and a system (2) for applying force including a plurality of support members (7), such as rollers, and drive elements for moving the support members against the tube in order to deform it against the pump body (3). The pump body (3) is movably mounted relative to the system (2) for applying force between a position spaced apart from the system (2) for applying force, and a position adjacent to the system (2) for applying force. The pump (1) also includes elements (36) for controlling the movement of the pump body (3) relative to the system (2) on the basis of a predetermined magnitude corresponding to the force applied to the tube.

In general, the present invention relates to peristaltic pumps.

More particularly and by way of example, the invention relates to a peristaltic pump for a dialysis machine, comprising firstly, a plate, known as a pump body, that includes a substantially plane surface against which a flexible tube for passing fluid is to be positioned, and secondly, a force application system comprising a plurality of presser members, such as rollers, and drive means for moving said presser members enabling said presser members to be moved while pressed against the tube in order to deform it against said pump body.

It has been observed that with certain peristaltic pumps, the flexible tube may not be pinched sufficiently between the pump body and the force application system, and that prevents fluid from flowing in the tube with sufficient flow rate and/or regularity. Conversely, with other pumps, the flexible tube is pinched too tightly between the pump body and the force application system, and that may also impede the flow of the fluid and may damage the tube.

Documents GB 2 230 301, GB 953 579, BE 685 301, and EP 0 837 242 are known, and they describe pumps each including a portion for supporting a tube for passing fluid that is movable relative to a force application system. The movement allowed between the portion for supporting the tube for passing fluid and the force application system is used to clear a passage so as to enable the tube to be inserted therein or in order to decrease or increase the contact surface area between the force application system and the tube in order to reduce or to increase the flow of fluid in the tube. However, the relative positioning between the pump body and the force application system remains difficult to achieve in reliable, accurate, and repeatable manner. Moreover, such pumps do not prevent the relative positioning between the pump body and the application system from being ill-adapted to the tube, thus leading to a risk of damaging the tube. Document FR 2 594 496 relates to a fluid transfer duct presenting an intermediate portion having a section that is larger and a wall of thickness that is considerably smaller, with resistance to deformation that is substantially lower in the other portions of said duct.

An object of the present invention is to provide a peristaltic pump making it possible to overcome the above-mentioned problems.

To this end, the invention provides a peristaltic pump for a dialysis machine, comprising firstly, a plate, known as a pump body, that includes a substantially plane surface against which a flexible tube for passing fluid is to be positioned, and secondly, a force application system comprising a plurality of presser members, such as rollers, and drive means for moving said presser members enabling said presser members to be moved while pressed against the tube in order to deform it against said pump body, said pump body being mounted to move relative to the force application system between a position spaced apart from said force application system and a position close to said force application system;

said pump being characterized in that it includes means for determining a magnitude representative of the force applied to the tube, when said tube is in its state positioned between the force application system and the pump body; and

in that said pump further comprises governor means for governing the movement of the pump body relative to the system as a function of said determined magnitude.

Said determined force corresponds to the pinching force to which the tube is subjected when engaged between the force application system and said pump body when one is moved towards the other. Taking account of the force applied to the tube in order to govern movement of the pump body relative to the force application system makes it possible for the pump body to pass from a position in which it is spaced apart from the force application system and in which the flexible tube may be freely positioned in order to easily insert it into the pump, to a close position in which the tube is pinched with a pinching force that is adapted automatically by measuring the force applied to the tube, so that said pinching force is sufficient to enable the force application system to deform it, and thus to cause the fluid to flow effectively, while being limited so as not to damage the tube. Said measured force is mainly transverse, preferably orthogonal, relative to the axis of the tube. Advantageously, this force measurement is performed in the absence of fluid flowing through the duct, i.e. when the elements of the force application system that are used to cause the fluid to flow, e.g. cylinders, are stationary (relative to one another).

Said force is measured at a given frequency until reaching the setpoint value, or a value contained in a range given around the setpoint value, in order to be able to govern the movement of the pump body relative to the force application system in real time.

The choice of measuring the pinching force so as to govern the movement of the pump body relative to the force application system is particularly advantageous in order to ensure a fluid flow rate through the tube that is sufficient, while reducing the risk of damage to the tube, since by means of this applied force parameter, a given degree of pinching may be obtained accurately, in reliable and repeatable manner, obtained for tubes of different diameters or materials.

In particular, governing the movement of the pump body as a function of the pinching force applied to the tube makes it possible to adapt the pinching force of the tube automatically in order to reach a setpoint value so that said force is sufficient to enable the force application system to deform the tube, and thus to cause the fluid to flow effectively, while being limited so as not to damage the tube and to allow effective flow of the fluid.

Designing a pump in this way makes it easier to install the tube in the pump body, e.g. a single use bloodline.

According to an advantageous characteristic of the invention, said pump body is mounted to move in translation along a direction that is transverse (preferably orthogonal) relative to said plane surface of the pump body.

According to an advantageous characteristic of the invention, said pump includes motor means for moving said pump body between said close position and said spaced apart position.

According to an advantageous characteristic of the invention, said governor means include means for defining a setpoint value for said magnitude, and regulator means that are configured to regulate the movement of the pump body in the direction moving the force application system closer or further apart as a function of the determined value of said magnitude in order to reach said setpoint value.

According to an advantageous characteristic of the invention, said means for determining a magnitude representative of the force applied to the tube, comprise at least one strain gauge, and preferably a plurality of strain gauges.

According to an advantageous characteristic of the invention, said means for driving the presser members in movement include a loop element that connects the presser members to one another, and two rotary cylinders, positioned inside and at opposite ends of said loop element, at least one of the cylinders being motor-driven in order to drive the loop element in movement around said cylinders.

According to an advantageous characteristic of the invention, said pump includes a housing in which the system and the pump body are housed, and the pump body is mounted to move relative to said housing.

According to an advantageous characteristic of the invention, said pump includes a housing in which the system and the pump body are housed, and said pump includes a diaphragm that, when in the inserted state inside the tube between the force application system and the pump body, extends around the peripheral wall of said tube in order to form a protective casing around said tube in cooperation with a wall of the housing.

The flexible leakproof diaphragm makes it possible to isolate the inside of the pump from the tube, and that makes it possible to avoid soiling the inside of the machine in the event of a leak. Furthermore, the leakproof flexible diaphragm also improves the soundproofing of the pump.

According to an advantageous characteristic of the invention, said diaphragm is made out of a liquid-proof flexible material.

The invention can be readily understood on reading the following description of embodiments, given with reference to the accompanying drawings, in which:

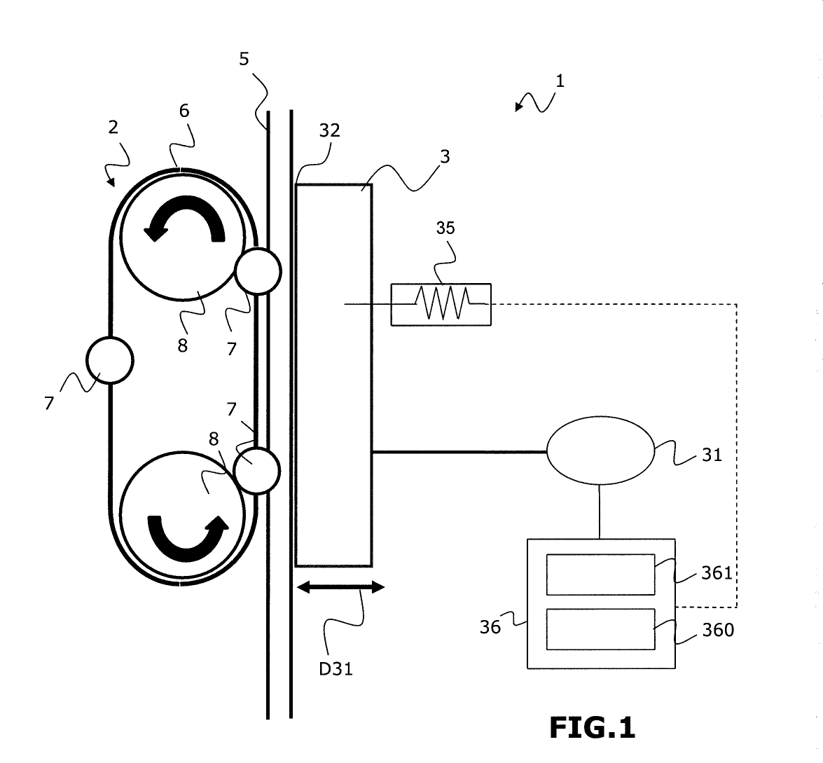

FIG. 1 is a diagrammatic view of a linear peristaltic pump in an embodiment of the invention;

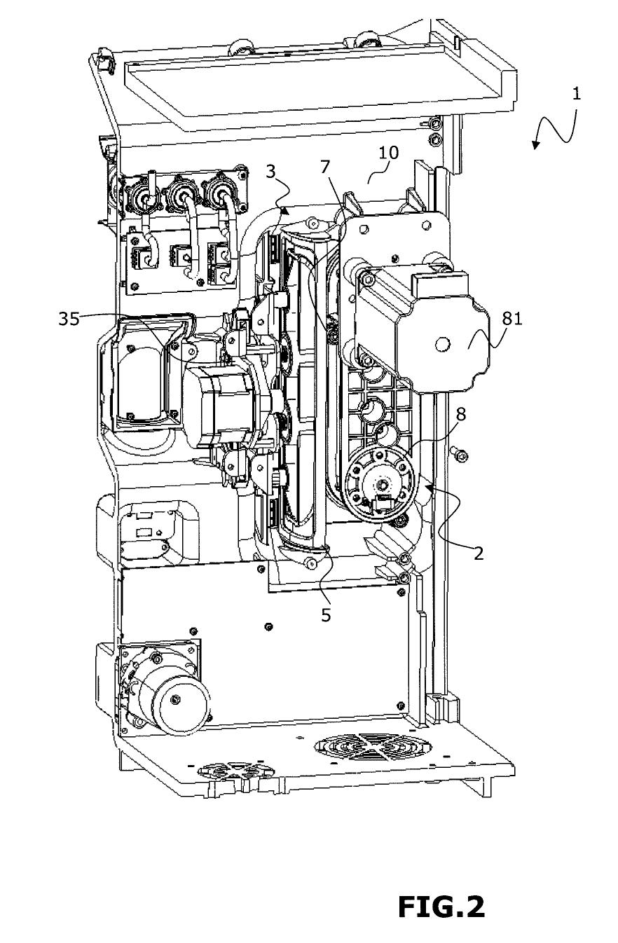

FIG. 2 is a perspective view of the inside of a portion of the FIG. 1 pump;

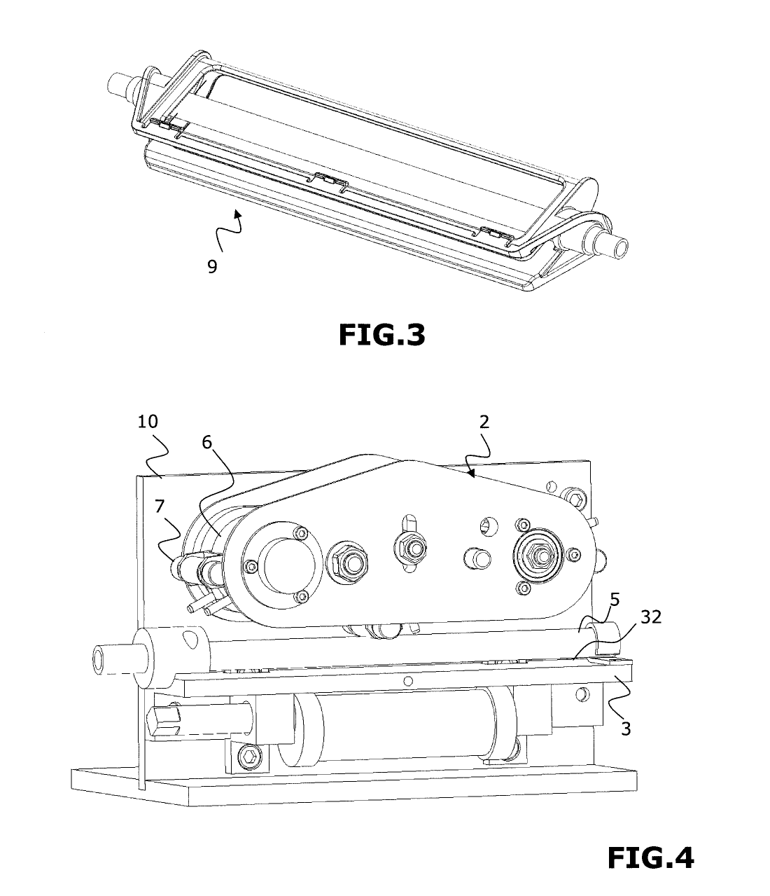

FIG. 3 is a perspective view of a leakproof diaphragm for surrounding the tube inserted into the FIG. 1 pump;

FIG. 4 is a perspective view of the pump body and of the force application system of the FIG. 1 pump in a spaced apart position so as to allow insertion of said tube; and

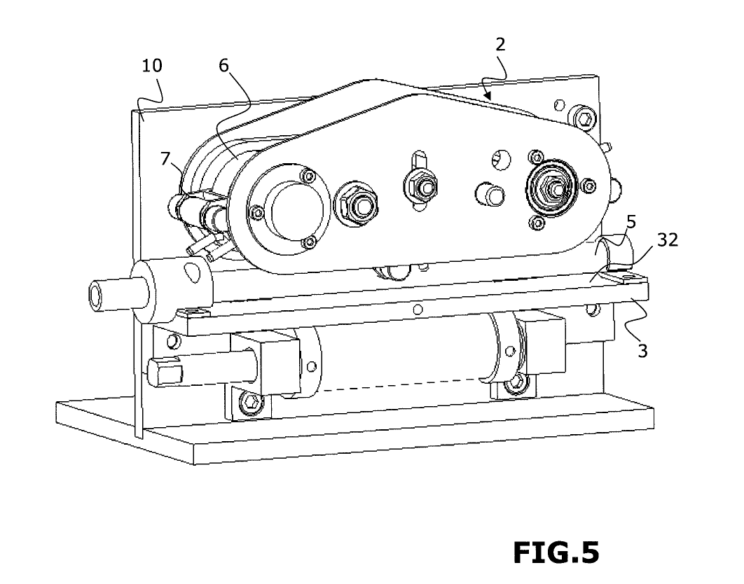

FIG. 5 is a perspective view of the pump body and of the force application system of the FIG. 1 pump in the close position.

With reference to the figures and as mentioned above, the invention relates to a linear peristaltic pump 1 for a dialysis machine. Said peristaltic pump 1 comprises a plate, known as a pump body 3, that includes a substantially plane surface 32 against which a flexible tube 5 for passing fluid is to be positioned. Said surface 32 is considered to be plane, or flat, i.e. straight, as opposed to contact surfaces in contact with the tube of so-called “rotary” peristaltic pump bodies that present a circularly arcuate shape.

The peristaltic pump 1 also comprises a force application system 2, the system having a plurality of presser members 7 and drive means for moving said presser members 7 enabling said presser members to be moved while pressed against the tube in order to deform it against said pump body 3.

Said pump body 3 and said system 2 are arranged relative to each other so as to allow a flexible tube 5 for passing fluid to be positioned between the plane surface 32 of the pump body 3 and the system 2. Said presser members 7 are moved along a closed loop path as described below. This closed loop path includes a portion directed towards the side of the pump body 3 and that allows the presser members 7 passing along this portion to press the duct against the plane surface 32 of the pump body 3.

Preferably, said pump is used for pumping blood in a dialysis machine. In this event, said tube 5 forms a bloodline.

Said plane surface 32 is directed towards said force application system 2 in order to make it possible for said system to apply a pressure force to the peripheral wall of said flexible tube 5 positioned against the pump body 3.

Said pump body 3 is mounted to move relative to the force application system 2 between a position spaced apart from said force application system (see FIG. 4), and a position close to said force application system 2 (see FIG. 5). Said pump body 3 and the force application system 2 thus define between them a space for inserting the substantially straight flexible tube since it extends along the plane surface 32 of the pump body.

When said force application system 2 is in a position spaced apart from the pump housing, the space left free between the force application system 2 and the pump body is sufficient for freely inserting the tube 5 between the force application system 2 and the pump body 3. Once the tube 5 is positioned against the plane face 32 of the pump body 3, the pump body 3 and the system 2 are brought into the close position in order to make it possible for the system 2 to apply force to the peripheral wall of the tube 5 from one end of the space for inserting the tube towards the other end.

A force is applied to the peripheral wall of the tube 5 by said presser members 7 pressing against the peripheral wall of said tube so as to gradually deform it along its portion pressing against the plane face 32 of the pump body 3 and thus causing the fluid contained in the tube 5 to flow from one end of the surface 32 towards its other end. Said pump body 3 is mounted to move in translation along a (preferably orthogonal) direction D31 that is transverse to said plane surface 32 of the pump body.

Said pump includes motor means 31 for moving said pump body 3 between said closer position and said spaced apart position.

Said pump 1 also includes means 35 for determining a magnitude that is representative of the force applied to the peripheral wall of the tube 5, when said tube 5 is in its state positioned between the force application system 2 and the pump body 3. Said means 35 for determining a magnitude representative of the force applied to the tube 5 include at least one strain gauge. Advantageously, said at least one strain gauge is positioned on a plate arranged so as to become deformed as a function of the relative position between system 2 and said pump body 3. Preferably, said means 35 include two or four strain gauges.

Said pump 1 further comprises governor means 36 for governing the movement of the pump body 3 relative to the system 2 as a function of said determined magnitude.

Said governor means are formed by an electronic and computer unit for processing and calculation. Said unit may be embodied in the form of an electronic circuit provided with a microcontroller or a microprocessor associated with a memory for storing data. Thus, in the description below, when it is specified that given means are configured to perform a given operation, it should be understood that the electronic and computer system forming said means, includes computer instructions making it possible to perform said operation.

Said governor means 36 include means 360 for defining a setpoint value for said magnitude. This setpoint value corresponds to the force desired for pinching the tube 5 between the system 2 and the pump body 3. Said governor means 36 further include regulator means 361 that are configured to regulate the movement of the pump body 3 in the direction moving the force application system 2 closer or further apart as a function of the determined value of said magnitude in order to reach said setpoint value.

Thus, using the determination means 35, said regulation means 36 are configured to acquire the value of said magnitude representative of the force applied to the peripheral wall of the tube 5, and to compare this acquired value with the stored setpoint value. Depending on the result of this comparison the governor means 36 control the motor 31 so that it moves the pump body 3 towards or away from the system 2 until a value is reached that is close to the setpoint value or that is located in a given range relative to the setpoint value.

Said drive means for moving the presser members 7 include a loop element 6 that connects the presser members 7 to one another, and two rotary cylinders 8, positioned inside and at opposite ends of said loop element 6. At least one of the cylinders 8 is motor-driven in order to cause the loop element 6 to move around said cylinders 8. Advantageously, the presser members 7 are rollers.

The loop element 6 comprises a drive belt arranged in a loop around the cylinders 8. One of the cylinders 8 is driven by a motor 81 so that said cylinder forms a cylinder suitable for driving the belt around said cylinders. The other cylinder forms a support for guiding movement of the belt.

The rollers 7 are mounted to be constrained to move with the belt 6 and they project from the outer face of said belt so that the tube is pressed when said rollers move along the tube.

As explained above, the loop element includes at least one substantially straight portion that extends substantially parallel to the plane surface 32 of the pump body 3, so that the rollers that are moved along said straight portion press the tube from one end of the portion towards the other end.

Said pump includes a housing 10 in which the system 2 and the pump body 3 are housed, and the pump body 3 is mounted to move relative to said housing 10.

As shown more particularly in FIG. 3, said pump 1 includes a diaphragm 9 that, when in the inserted state inside the tube 5 between the force application system 2 and the pump body 3, extends around the peripheral wall of said tube 5 in order to form a protective casing around said tube 5 in cooperation with a wall of the housing 10, opposite the bottom of the diaphragm.

Said diaphragm 9 is made out of a liquid-proof flexible material.

Advantageously, provision may be made for the space left free between the system 2 and the pump body 3 to be located in the top portion of the pump so as to be easily accessible by the operator.

The present invention is not limited in any way to the embodiments described and shown, and the person skilled in the art can make any variation thereto in accordance with its spirit.

1-9. (canceled)

10. A peristaltic pump (1), for example for a dialysis machine, comprising firstly, a plate, known as a pump body (3), that includes a substantially plane surface (32) against which a flexible tube (5) for passing fluid is to be positioned, and secondly, a force application system (2) comprising a plurality of presser members (7), such as rollers, and drive means for moving said presser members (7) enabling said presser members to be moved while pressed against the tube in order to deform it against said pump body (3);
said pump body (3) being mounted relative to the force application system (2) between a position spaced apart from said force application system (2) and a position close to said force application system (2);
said pump (1) being characterized in that it includes means (35) for determining a magnitude representative of the force applied to the tube (5), when said tube (5) is in the state positioned between the force application system (2) and the pump body (3);
in that said pump (1) further comprises governor means (36) for governing the movement of the pump body (3) relative to the system (2) as a function of said determined magnitude; and
in that said governor means (36) include means (360) for defining a setpoint value for said magnitude, and regulator means (361) that are configured to regulate the movement of the pump body (3) in the direction moving the force application system (2) closer or further apart as a function of the determined value of said magnitude in order to reach said setpoint value.

11. A pump (1) according to claim 10, characterized in that said pump body (3) is mounted to move in translation along a direction that is transverse, preferably orthogonal, relative to said plane surface (32) of the pump body (3).

12. A pump (1) according to claim 10, characterized in that said pump (1) includes motor means (31) for moving said pump body (3) between said close position and said spaced apart position.

13. A pump (1) according to claim 10, characterized in that said means (35) for determining a magnitude representative of the force applied to the tube (5), comprise at least one strain gauge, and preferably a plurality of strain gauges.

14. A pump (1) according to claim 10, characterized in that said drive means for moving the presser members (7) include a loop element (6) that connects the presser members (7) to one another, and two rotary cylinders (8), positioned inside and at opposite ends of said loop element (6), at least one of the cylinders (8) being motor-driven in order to drive movement of the loop element (6) around said cylinders (8).

15. A pump (1) according to claim 10, characterized in that said pump includes a housing (10) in which the system (2) and the pump body (3) are housed; and in that the pump body (3) is mounted to move relative to said housing (10).

16. A pump (1) according to claim 10, characterized in that said pump includes a housing (10) in which the system (2) and the pump body (3) are housed; and
in that said pump (1) includes a diaphragm (9) that, when in the inserted state inside the tube (5) between the force application system and the pump body (3), extends around the peripheral wall of said tube (5) in order to form a protective casing around said tube (5) in cooperation with a wall of the housing (10).

17. A pump (1) according to claim 16, characterized in that said diaphragm (9) is made out of a liquid-proof flexible material.

18. A pump (1) according to claim 11, characterized in that said pump (1) includes motor means (31) for moving said pump body (3) between said close position and said spaced apart position.

19. A pump (1) according to claim 11, characterized in that said means (35) for determining a magnitude representative of the force applied to the tube (5), comprise at least one strain gauge, and preferably a plurality of strain gauges.

20. A pump (1) according to claim 11, characterized in that said drive means for moving the presser members (7) include a loop element (6) that connects the presser members (7) to one another, and two rotary cylinders (8), positioned inside and at opposite ends of said loop element (6), at least one of the cylinders (8) being motor-driven in order to drive movement of the loop element (6) around said cylinders (8).

21. A pump (1) according to claim 11, characterized in that said pump includes a housing (10) in which the system (2) and the pump body (3) are housed; and in that the pump body (3) is mounted to move relative to said housing (10).

22. A pump (1) according to claim 11, characterized in that said pump includes a housing (10) in which the system (2) and the pump body (3) are housed; and
in that said pump (1) includes a diaphragm (9) that, when in the inserted state inside the tube (5) between the force application system and the pump body (3), extends around the peripheral wall of said tube (5) in order to form a protective casing around said tube (5) in cooperation with a wall of the housing (10).
